(19) United States
(12) Patent Application Publication
(10) Pub. No.: US 2025/0009841 A1
(43) Pub. Date: Jan. 9, 2025

(54) Methods of Diagnosing and Managing Bleeding Disorders

(71) Applicant: Emory University, Atlanta, GA (US)

(72) Inventors: Wilbur A. Lam, Atlanta, GA (US); Oluwamayokun Oshinowo, Atlanta, GA (US); David R. Myers, Atlanta, GA (US); Carolyn M. Bennett, Atlanta, GA (US); Renhao Li, Atlanta, GA (US)

(21) Appl. No.: 18/766,306

(22) Filed: Jul. 8, 2024

Related U.S. Application Data
(60) Provisional application No. 63/525,568, filed on Jul. 7, 2023.

(51) Int. Cl. A61K 38/17; A61P 7/06; G01N 15/01

(52) U.S. Cl. CPC A61K 38/1709; A61P 7/06; G01N 15/01; G01N 2333/75

(57) ABSTRACT

In certain embodiment, this disclosure relates to methods of treating, preventing, or aiding in the diagnosis of a patient having a risk of excessive bleeding. In certain embodiment, the patient is at risk of excessive bleeding due to a platelet disorder or autoimmune disorder. In certain embodiments, this disclosure relates to treatment methods comprising administering an effective amount of an agent that binds specific segments of the alpha subunit or beta subunit of an αIIbβ3 integrin complex to a subject in need thereof.

### CROSS-REFERENCE TO RELATED APPLICATIONS

This application claims the benefit of U.S. Provisional Application No. 63/525,568 filed Jul. 7, 2023. The entirety of this application is hereby incorporated by reference for all purposes.

### STATEMENT REGARDING FEDERALLY SPONSORED RESEARCH OR DEVELOPMENT

This invention was made with government support under HL155330, HL145000, and HL160210 awarded by the National Institutes of Health. The government has certain rights in the invention.

### BACKGROUND

Immune thrombocytopenia purpura (ITP) is an autoimmune disease that results in an inadequate production of platelets often leading to bleeding complications. ITP is clinically diagnosed as a platelet count less than 100×10⁹/L with other causes of thrombocytopenia excluded. For the individuals affected by ITP, the clinical challenge lies in predicting which of the minority of patients need immediate initiation of therapy due to risk of life-threatening bleeding. While ITP patients have an increased bleeding risk, due in part to the low circulating platelet concentrations, a subset of patients are at risk for severe bleeding. For example, 20% of patients are at risk for major bleeding episodes and 1 in 200 are at risk for life threatening hemorrhage. No biomarker objectively and reliably correlates with bleeding to guide treatment and predict clinical outcomes. Even low platelet counts, no matter how extreme, only loosely correlate with risk and severity of bleeding. This lack of a biomarker is problematic as the mainstay of ITP therapies involve immunosuppression and/or splenectomy, which harbor significant side effects. Taken together, clinicians are forced to balance the potential increased bleeding risk with significant side effects of current therapies, which may not be necessary since the majority of patients spontaneously resolve without intervention. Thus, there is a need to identify improved methods for diagnosing and treating patients with ITP.
Oshinowo et al. report platelet contraction force as a biophysical biomarker for bleeding risk in patients with immune thrombocytopenia. Blood, 2018, 132 (Suppl_1): 517.
Oshinowo et al. report increased platelet force is associated with decreased bleeding severity in pediatric immune thrombocytopenia. Blood, 2019, 134 (Supplement_1): 1096.
Myers et al. report single-platelet nanomechanics measured by high-throughput cytometry. Nat Mater, 2017, 16 (2): 230-235. See also U.S. Pat. No. 10,168,341.
Sang et al. provide a review on the interplay between platelets and coagulation. Blood Reviews, 2021, 46 100733.
Kerkhof et al. report a review on exogenous integrin IIb3 Inhibitors. J Mol Sci, 2021, 22, 3366.
Frelinger et al. report monoclonal antibodies to ligand-occupied conformers of integrin glycoprotein IIb-IIIa alter receptor affinity, specificity, and function. J Biol Chem. 1991, 266(26): 17106-11.

References cited herein are not an admission of prior art.

### SUMMARY

In certain embodiment, this disclosure relates to methods of treating, preventing, or aiding in the diagnosis of a patient having a risk of excessive bleeding. In certain embodiments, the patient is at risk of excessive bleeding due to a platelet disorder or autoimmune disorder. In certain embodiments, this disclosure relates to treatment methods comprising administering an effective amount of an agent that binds a specific domain of the alpha subunit of an αIIbβ3 integrin complex or a specific domain of the beta subunit of an αIIbβ3 integrin complex to a subject in need thereof.

In certain embodiments, this disclosure relates to methods of identifying, diagnosing, and treating a subject that is at a high risk of serious bleeding episodes, risk for severe bleeding, risk for major bleeding episodes, or at risk for life threatening hemorrhage using methods disclosed herein and treating a patient that is at risk of excessive bleeding with an agent that improves the ability of platelets to coagulate or form blood clots. In certain embodiments, an agent that improves the ability of platelets to coagulate or form blood clots is an agent that binds the Caf 1 domain or Caf 2 domain of the alpha subunit of an αIIbβ3 integrin complex or the membrane-proximal-tail domain or any of the four epidermal growth factor (EGF)-like domains of the beta subunit of an αIIbβ3 integrin complex to a subject in need thereof.

In certain embodiments, this disclosure relates to methods of increasing blood clotting comprising administering an effective amount of an agent that binds a Caf 1 domain or Caf 2 domain of the alpha subunit of an αIIbβ3 integrin complex to a subject in need thereof.

In certain embodiments, the subject is diagnosed with a low platelet count and a low platelet contraction force. In certain embodiments, the subject is diagnosed with a low contraction force using a contraction cytometer.

In certain embodiments, the subject is diagnosed with an autoimmune disease or inflammatory disorder. In certain embodiments, the subject is diagnosed with immune thrombocytopenia (ITP).

In certain embodiments, the agent is an antibody. In certain embodiments, the agent is an MBC.314.5 antibody or fragment thereof. In certain embodiments, the agent is administered in combination with a blood clotting agent. In certain embodiments, the agent is administered in combination with thrombin, fibrinogen, aprotinin, tranexamic acid (TXA), epsilon-aminocaproic acid, 4-(aminomethyl)benzoic acid, or combinations thereof. In certain embodiments, the agent is administered topically in a composition further comprising a clotting agent, gelatin, cellulose, collagen, chitosan, aluminosilicate, albumin, thrombin, fibrinogen, or combinations thereof.

In certain embodiments, this disclosure relates to methods of increasing blood clotting comprising administering an effective amount of an agent that binds a membrane-proximal-tail domain or any of the four epidermal growth factor (EGF)-like domains of the beta subunit of an αIIbβ3 integrin complex to a subject in need thereof.

In certain embodiments, the subject is diagnosed with a low platelet count and a low platelet contraction force. In certain embodiments, the subject is diagnosed with a low contraction force using a contraction cytometer.

In certain embodiments, the subject is diagnosed with an autoimmune disease or inflammatory disorder. In certain embodiments, the subject is diagnosed with immune thrombocytopenia (ITP).

In certain embodiments, the agent is an antibody. In certain embodiments, the agent is an MBC 322.8, anti-LIBS-1, anti-LIBS-2 anti-LIBS3, anti-LIBS6 antibody, or fragment thereof. In certain embodiments, the agent is administered in combination with a blood clotting agent. In certain embodiments, the agent is administered in combination with thrombin, fibrinogen, aprotinin, tranexamic acid (TXA), epsilon-aminocaproic acid, 4-(aminomethyl)benzoic acid, or combinations thereof. In certain embodiments, the agent is administered topically in a composition further comprising a clotting agent, gelatin, cellulose, collagen, chitosan, aluminosilicate, albumin, thrombin, fibrinogen, or combinations thereof.

In certain embodiments, this disclosure relates to a platelet contraction cytometer comprising a micropatterned surface having pairs of first and second zones for use in measuring platelet contraction forces.

### BRIEF DESCRIPTION OF THE SEVERAL VIEWS OF THE DRAWINGS

FIGS. 1A-1C illustrate a platelet contraction cytometer used to perform high throughput measurements of single cell forces.

FIG. 1A illustrates construction of a cellular contraction cytometer comprising a microscope coverslip functionalized with a polyacrylamide hydrogel micropatterned with fluorescent extracellular matrix proteins.

FIG. 1B illustrates that individual platelets adhere, spread, and contract against the micropatterned fibrinogen microdot pairs on the hydrogel. The microdots enable calculations of platelet contractile forces at the single cell level using measurements of the microdot size and spacing. This micropatterned coverslip is adaptable for use with a variety of fluorescence microscopes. Large array of fibrinogen microdot pairs enables numerous images, and therefore, measurements of platelet contraction forces to be captured.

FIG. 1C shows a representative histogram from of a healthy adult, with a distribution of low, moderate, and highly contractile platelets.

FIGS. 2A-2C show data indicating platelet contraction force is a clinically relevant biophysical biomarker for bleeding risk in patients with immune thrombocytopenia (ITP).

FIG. 2A shows data indicating ITP patients with bleeding scores of 2-4 consistently lack highly contractile platelets as compared to patients with little or no bleeding.

FIG. 2B shows data indicating the average platelet force of patients with moderate to heavy bleeding were all significantly lower than patients with no bleeding symptoms. Platelet count, which is currently the main clinical biomarker used in ITP, no matter how low, loosely correlates with Buchanan bleeding scores, as a substantial population of patients have extremely low platelet counts (less than 20 k/μL), but little to no bleeding.

FIG. 2C shows data indicating platelet force best stratifies low bleeding score patients from higher bleeding score patients. When platelet force is considered in the context of platelet count and clinical bleeding severity score, platelet force and platelet count synergistically stratify patients by clinical severity, whereas patients with platelet counts less than 40 k/μL and platelet forces below 25 nN have an increased likelihood of having higher bleeding scores.

FIG. 3A shows data and illustrates that ITP Patient polyclonal IgG platelet-associated (PA) antibodies are directed towards epitopes the integrin αIIbβ3 and modulate platelet force in a confirmation-dependent manner. Contraction cytometry was performed on the platelets from ITP patients and polyclonal IgG antibodies were extracted from the plasma for further analysis. In those patients, the extent of clinical bleeding severity scores correlated with the lack of highly contractile platelets. Isolated IgG antibodies (10 μg/mL) from those ITP patients with bleeding modulated the forces of platelets from healthy donors (2-3 donors/condition, greater than 390 platelets/condition). As platelet contractile force is transmitted via the αIIbβ3 integrin, negative stain electron microscopy was used to evaluate those specific antibody-integrin interactions. Autoantibodies from ITP patients with higher bleeding scores stabilized integrins in the bent and extended closed conformations.

FIG. 3B shows data and illustrates that immuno-mechanical modulation by autoantibodies directed to specific αIIbβ3 integrin epitopes are associated with increases and decreases in platelet contractile force. To investigate the relationship between conformation- and epitope-dependent platelet contractile force modulation, a panel of well-characterized monoclonal antibodies (2.5 μg/mL) directed towards different epitopes of αIIbβ3 were used. Exposure to monoclonal antibodies alone can modulate platelet force by approximately plus or minus 15 nN (55%). Moreover, a pattern to the platelet contractile force modulation became evident, as antibodies that bind closer to the tail region increase force whereas antibodies that bind towards the head region decrease force (2-4 donors/condition, greater than 120 platelets/condition). Matching electron microscopy data from patient polyclonal autoantibodies, negative stain electron microscopy indicated that antibodies that cause a decrease in contractile force bound mostly to the bent conformation and extended closed formation of the integrin; however, this data showed that there is only a minor decrease in force when all integrins are stabilized in the extended closed conformation. Antibodies that stabilized integrins in an extended open conformation were associated with increased forces. Antibody treated platelets were compared to the untreated condition.

FIG. 4 illustrates the use of force cytometry to reveal single platelet contraction force indicated as a clinical biophysical biomarker for bleeding severity modulated by autoantibodies targeted to the αIIbβ3 platelet integrin. Micropatterned hydrogel-laden microscope coverslip-based cellular contraction cytometry technology demonstrates a direct link between platelet biophysics and a bleeding phenotype of a patient. ITP patients bleeding scores directly correlate with a decrease in single platelet contraction. Mechanistically, autoantibodies of a patient that target the αIIbβ3 integrin “immuno-mechanically” modulate platelet contractile forces (increasing or decreasing) in a conformation- and epitope-dependent manner. Antibodies that bind to the bent or extended-closed integrin conformation decrease platelet contractile force whereas antibodies that bind to the extended-open integrin conformation increase force. Therefore, platelet contractility correlates inversely with bleeding severity in that patient platelets with low contraction forces are associated with an increased risk of a bleeding phenotype, whereas a patient with highly contractile platelets will likely be asymptomatic.

FIG. 5 shows data from experiments where platelets treated with low force antibodies had extremely low platelet forces and lacked a highly contractile platelet population. However, platelets treated with the same concentration of low force antibodies, but varying concentrations of high force antibodies, had higher forces and an increasing percentage of high force platelets.

### DETAILED DISCUSSION

Before the present disclosure is described in greater detail, it is to be understood that this disclosure is not limited to particular embodiments described, and as such may vary. It is also to be understood that the terminology used herein is for the purpose of describing particular embodiments only, and is not intended to be limiting, since the scope of the present disclosure will be limited only by the appended claims or as amended during prosecution. Although the function of certain compositions disclosed herein are believed to operate by particular mechanisms, it is not intended that embodiments of this disclosure be limited by any specific mechanism.

Unless defined otherwise, all technical and scientific terms used herein have the same meaning as commonly understood by one of ordinary skill in the art to which this disclosure belongs. Although any methods and materials similar or equivalent to those described herein can also be used in the practice or testing of the present disclosure, the preferred methods and materials are now described.

All publications and patents cited in this specification are herein incorporated by reference as if each individual publication or patent were specifically and individually indicated to be incorporated by reference and are incorporated herein by reference to disclose and describe the methods and/or materials in connection with which the publications are cited.

As will be apparent to those of skill in the art upon reading this disclosure, each of the individual embodiments described and illustrated herein has discrete components and features which may be readily separated from or combined with the features of any of the other several embodiments without departing from the scope or spirit of the present disclosure. Any recited method can be carried out in the order of events recited or in any other order that is logically possible.

An “embodiment” of this disclosure refers to an example and infers that the disclosure is not necessarily limited to the example. Embodiments of the present disclosure will employ, unless otherwise indicated, techniques of medicine, organic chemistry, biochemistry, molecular biology, pharmacology, and the like, which are within the skill of the art. Such techniques are explained fully in the literature.

It must be noted that, as used in the specification and the appended claims, the singular forms “a,” “an,” and “the” include plural referents unless the context clearly dictates otherwise. In this specification and in the claims that follow, reference will be made to a number of terms that shall be defined to have the following meanings unless a contrary intention is apparent.

As used herein, the term “about” means a range of values including the specified value, which a person of ordinary skill in the art would consider reasonably similar to the specified value. In embodiments, about means within a standard deviation using measurements generally acceptable in the art. In embodiments, about means a range extending to plus or minus 10% of the specified value. In embodiments, about includes the specified value.

As used in this disclosure and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) have the meaning ascribed to them in U.S. Patent law in that they are inclusive or open-ended and do not exclude additional, unrecited elements or method steps.

“Consisting essentially of” or “consists of” or the like, have the meaning ascribed to them in U.S. Patent law in that when applied to methods and compositions encompassed by the present disclosure refers to the idea of excluding certain prior art element(s) as an inventive feature of a claim but may contain additional composition components or method steps, etc., that do not materially affect the basic and novel characteristic(s) of the compositions or methods, compared to those of the corresponding compositions or methods disclosed herein.

“Subject” refers to any animal, preferably a human patient, livestock, rodent, monkey, or domestic pet.

As used herein, the terms “prevent” and “preventing” include the prevention of the recurrence, spread or onset. It is not intended that the present disclosure be limited to complete prevention. In some embodiments, the onset is delayed, or the severity of the disease is reduced.

As used herein, the terms “treat” and “treating” are not limited to the case where the subject (e.g., patient) is cured and the disease is eradicated. Rather, embodiments of the present disclosure also contemplate treatment that merely reduces symptoms, and/or delays disease progression.

As used herein, the term “combination with” when used to describe administration with an additional treatment means that the agent may be administered prior to, together with, or after the additional treatment, or a combination thereof.

The term “effective amount” refers to that amount of a compound or pharmaceutical composition described herein that is sufficient to effect the intended application including, but not limited to, disease treatment as illustrated below. The therapeutically effective amount can vary depending upon the intended application (in vitro or in vivo), or the subject and disease condition being treated, e.g., the weight and age of the subject, the severity of the disease condition, the manner of administration and the like, which can readily be determined by one of ordinary skill in the art. The specific dose will vary depending on, for example, the particular compounds chosen, the dosing regimen to be followed, whether it is administered in combination with other agents, timing of administration, the tissue to which it is administered, and the physical delivery system in which it is carried.

In certain contexts, an “antibody” refers to a protein-based molecule that is naturally produced by animals in response to the presence of a protein or other molecule or that is not recognized by the animal's immune system to be a “self” molecule, i.e., recognized by the animal to be a foreign molecule, i.e., an antigen to the antibody. The immune system of the animal will create an antibody to specifically bind the antigen, and thereby targeting the antigen for degradation or elimination, or any cell or organism attached to the antigen. It is well recognized by skilled artisans that the molecular structure of a natural antibody can be synthesized and altered by laboratory techniques. Recombinant engineering can be used to generate fully synthetic antibodies or fragments thereof providing control over variations of the amino acid sequences of the antibody. Thus, the term “antibody” is intended to include natural antibodies, monoclonal antibody, or non-naturally produced synthetic antibodies, such as specific binding single chain antibodies, bispecific antibodies, or fragments thereof. These antibodies may have chemical modifications. The term “monoclonal antibodies” refers to a collection of antibodies encoded by the same nucleic acid molecule that are optionally produced by a single hybridoma (or clone thereof) or other cell line, or by a transgenic mammal such that each monoclonal antibody will typically recognize the same antigen. The term “monoclonal” is not limited to any particular method for making the antibody, nor is the term limited to antibodies produced in a particular species, e.g., mouse, rat, etc.

From a structural standpoint, an antibody is a combination of proteins: two heavy chain proteins and two light chain proteins. The heavy chains are longer than the light chains. The two heavy chains typically have the same amino acid sequence. Similarly, the two light chains typically have the same amino acid sequence. Each of the heavy and light chains contain a variable segment that contains amino acid sequences which participate in binding to the antigen. The variable segments of the heavy chain do not have the same amino acid sequences as the light chains. The variable segments are often referred to as the antigen binding domains. The antigen and the variable regions of the antibody may physically interact with each other at specific smaller segments of an antigen often referred to as the “epitope.” Epitopes usually consist of surface groupings of molecules, for example, amino acids or carbohydrates. The terms “variable region,” “antigen binding domain,” and “antigen binding region” refer to that portion of the antibody molecule which contains the amino acid residues that interact with an antigen and confer on the antibody its specificity and affinity for the antigen. Small binding regions within the antigen-binding domain that typically interact with the epitope are also commonly alternatively referred to as the “complementarity-determining regions, or CDRs.”

With regard to variable chain immunoglobulins, the location of binding complementarity-determining regions (CDRs) sometimes varies depending on the specific sequence context and animal. The CDRs can be determined through epitope studies and sequence alignment comparisons of the constant and framework regions for the specific animal. As is well-known in the art that there are multiple conventions to define and describe the CDRs of a VH or VHH fragment, such as the Kabat definition (which is based on sequence variability) and the Chothia definition (which is based on the location of the structural loop regions).

In general, identifying CDRs can be accomplished utilized the following rules using Kabat or Chothia antibody sequence criteria. Note that there are examples where these constant features do not occur; however, the Cys residues are the most common conserved feature.

For CDR-L1, the start residue is approximately 24 to 30 after the first amino acid and typically after a Cys. The residue after is typically a Trp such as Trp-Tyr-Gln, Trp-Leu-Gln, Trp-Phe-Gln, or Trp-Tyr-Leu. Length is typically 10 to 17 residues. CDR-L2 starts about 16 residues after the end of L1. The residues before are typically Ile-Tyr, Val-Tyr, Ile-Lys, Ile-Phe, with a length of about 4-7 residues. CDR-L3 starts at about 33 residues after end of L2 before a Cys residue with a length of about 7 to 11 residues typically ending before a Phe-Gly.

For CDR-H1, the start residue is approximately 26 to 30 after the first amino acid and typically 4 amino acids after a Cys and typically ends with Trp, e.g., Trp-Val, but also, Trp-Ile, Trp-Ala. The length is typically about 6 to 12 residues. CDR-H2 typically starts at about 4-15 residues after the end of CDR-H1. Residues before the start are typically Trp-Ile-Gly but can be a number of variations, and residues after typical ends with Lys/Arg-Leu/Ile/Val/Phe/Thr/Ala-Thr/Ser/Ile/Ala. The length can vary from about 8 to 20 amino acids; CDR-H3 is typically about 30-33 residues after the end of CDR-H2, and often identified 3 amino acids after a Cys, such as in the example Cys-Ala-Arg. The end is sometimes identified before residues such as Trp-Gly. The length can vary widely, e.g., 4-25 or more depending on the animal.

A “chimeric antibody” is a molecule in which different portions of the antibody are derived from different immunoglobulin molecules such that the entire molecule is not naturally occurring. Examples of chimeric antibodies include those having a variable region derived from a non-human antibody and a human immunoglobulin constant region. The term is also intended to include antibodies having a variable region derived from one human antibody grafted to an immunoglobulin constant region of a predetermined sequences or the constant region from another human for which there are allotypic differences residing in the constant regions of any naturally occurring antibody having the variable regions, e.g., CDRs 1, 2, and 3 of the light and heavy chain. Human heavy chain genes exhibit structural polymorphism (allotypes) that are inherited as a haplotype. The serologically defined allotypes differ within and between population groups. Sec Jefferis et al. mAb, 1 (2009), pp. 332-338. In certain embodiments, the antibody, antigen binding fragment, the light chain, or the heavy chain comprises a non-naturally occurring chimeric amino acid sequence such that there is at least one mutation that is not present in naturally occurring antibodies comprising one or all of the six CDRs.

The term “antibody fragment” refers to an antibody which comprises less than a complete, intact antibody. Complete antibodies comprise two functionally independent parts or fragments: an antigen binding fragment known as “Fab,” and a carboxy terminal crystallizable fragment known as the “Fc” fragment. The Fab fragment includes the first constant domain from both the heavy and light chain (CH1 and CL1) together with the variable regions from both the heavy and light chains that bind the specific antigen. Each of the heavy and light chain variable regions includes three complementarity determining regions (CDRs) and framework amino acid residues which separate the individual CDRs. The Fc region comprises the second and third heavy chain constant regions (CH2 and CH3) and is involved in effector functions such as complement activation and attack by phagocytic cells. In some antibodies, the Fc and Fab regions are separated by an antibody “hinge region,” and depending on how the full-length antibody is proteolytically cleaved, the hinge region may be associated with either the Fab or Fc fragment. For example, cleavage of an antibody with the protease papain results in the hinge region being associated with the resulting Fc fragment, while cleavage with the protease pepsin provides a fragment wherein the hinge is associated with both Fab fragments simultaneously. Because the two Fab fragments are covalently linked following pepsin cleavage, the resulting fragment is termed the F(ab′)2 fragment.

The term, “humanized” refers to an antibody containing one or more amino acid mutations so that immunogenicity upon administration in human patients, e.g., due to “pre-existing antibodies”, is reduced, made highly unlikely, or nonexistent. Anaphylaxis is a severe allergic reaction to an allergen, e.g., polypeptide. Non-human proteins contain amino acid residues that may be immunogenic when targeted by preexisting antibodies circulating in a human patient. Thus, it is desirable to mutate residues within a therapeutic antibody so that the peptide sequences are similar to peptide sequences that commonly occurs in human antibodies/proteins, provided that the desirable therapeutic properties are retained, thereby reducing the risk of undesirable allergic reactions. In antibodies, this is typically accomplished by transfer of complementarity-determining regions (CDRs) of a non-human antibody to a human framework sequence, yielding a human like antibody with reduced immunogenicity. Another method entails comparing sequences, preferably framework sequences, and identifying amino acid substitutions providing “humanized” sequences frequently found within human antibody sequence repertoire. These humanized sequences reduce the risk of undesirable immune reactions providing an antibody that is substantially non-immunogenic in humans and retain the affinity and activity of the original polypeptide. In one example humanization, e.g., framework region humanization, one screens the available human consensus sequences for existing known sequences that are most similar to the original sequence. Using computer databases to compare human consensus frameworks for humanizing antibodies is well-known. See e.g., A universal combinatorial design of antibody framework to graft distinct CDR sequences: a bioinformatics approach, Proteins, 2012, 80(3): 896-912 and Clavero-Álvarez et al. Humanization of Antibodies using a Statistical Inference Approach, Scientific Reports, 2018, volume 8, Article number: 14820. European Patent No. EP 239,400; International Publication No. WO 91/09967; and U.S. Pat. Nos. 5,225,539, 5,530,101, and 5,585,089, veneering or resurfacing (European Patent Nos. EP 592,106 and EP 519,596; Padlan, 1991, Molecular Immunology 28(4/5): 489-498; Studnicka et al., 1994, Protein Engineering 7(6): 805-814; and Roguska et al., 1994, Proc. Natl. Acad. Sci. 91:969-973), chain shuffling (U.S. Pat. No. 5,565,332), and techniques disclosed in, e.g., U.S. Pat. Nos. 6,407,213, 5,766,886, 5,585,089, International Publication No. WO 9317105, Tan et al., 2002, J. Immunol. 169:1119-25, Caldas et al., 2000, Protein Eng. 13:353-60, Morca et al., 2000, Methods 20:267-79, Baca et al., 1997, J. Biol. Chem. 272:10678-84, Roguska et al., 1996, Protein Eng. 9:895-904, Couto et al., 1995, Cancer Res. 55(23 Supp): 5973s-5977s, Couto et al., 1995, Cancer Res. 55:1717-22, Sandhu, 1994, Gene 150:409-10, Pedersen et al., 1994, J. Mol. Biol. 235:959-73, Jones et al., 1986, Nature 321:522-525, Riechmann et al., 1988, Nature 332:323, and Presta, 1992, Curr. Op. Struct. Biol. 2:593-596.

The framework and CDR regions of a humanized antibody need not correspond precisely to the parental sequences, e.g., the donor CDR or the consensus framework may be mutagenized by substitution, insertion, or deletion of at least one residue so that the CDR or framework residue at that site does not correspond exactly to either the consensus or the donor antibody. Such mutations, however, are preferably not extensive. Usually, at least 75% of the humanized antibody residues will correspond to those of the parental framework region (FR) and CDR sequences, more often 90%, and most preferably greater than 95%.

In certain instances, framework residues in the framework regions will be substituted with the corresponding residue from the CDR donor antibody to alter, preferably improve, antigen binding. These framework substitutions are identified by methods well known in the art, e.g., by modeling of the interactions of the CDR and framework residues to identify framework residues important for antigen binding and sequence comparison to identify unusual framework residues at particular positions. (Sec, e.g., Queen et al., U.S. Pat. No. 5,585,089; U.S. Publication Nos. 2004/0049014 and 2003/0229208; U.S. Pat. Nos. 6,350,861; 6,180,370; 5,693,762; 5,693,761; 5,585,089; and 5,530,101 and Riechmann et al., 1988, Nature 332:323).

A “label” refers to a detectable compound or composition that is conjugated directly or indirectly to another molecule, such as an antibody or a protein, to facilitate detection of that molecule. Specific, non-limiting examples of labels include fluorescent tags, enzymatic linkages, and radioactive isotopes. A label includes the incorporation of a radiolabeled amino acid or the covalent attachment of biotinyl moieties to a polypeptide that can be detected by marked avidin (for example, streptavidin containing a fluorescent marker or enzymatic activity that can be detected by optical or colorimetric methods). Various methods of labeling polypeptides and glycoproteins are known in the art and may be used. Examples of labels for polypeptides include, but are not limited to, the following: radioisotopes or radionucleotides (such as ³⁵S or ¹³¹I) fluorescent labels (such as fluorescein isothiocyanate (FITC), rhodamine, lanthanide phosphors), enzymatic labels (such as horseradish peroxidase, beta-galactosidase, luciferase, alkaline phosphatase), chemiluminescent markers, biotinyl groups, predetermined polypeptide epitopes recognized by a secondary reporter (such as a leucine zipper pair sequences, binding sites for secondary antibodies, metal binding domains, epitope tags), or magnetic agents, such as gadolinium chelates. In some embodiments, labels are attached by spacer arms of various lengths to reduce potential steric hindrance.

As used herein, methods for “aiding diagnosis” or “assisting in diagnosis” both refer to methods that assist in making a clinical determination regarding the presence or progression of an adverse bleeding event and may or may not be conclusive with respect to the definitive diagnosis.

As used herein, the term “predicting” refers to making a finding with notably enhanced likelihood of developing an adverse bleeding event.

The term “sample” is used in its broadest sense, in that it has chemical makeup that is physical for analysis, i.e., analyte. In one sense it can refer to a nasal fluid, saliva, cough droplets, or expelled droplets of saliva into the air, e.g., produced by speaking, or other lung fluid or blood. In another sense, it is meant to include a specimen or culture obtained from any source, as well as biological and environmental samples. Biological samples include bodily fluids, urine, feces, nasal drip, seminal fluid, hair, skin (dead or epithelial layer of skin), finger or toenail clipping, and a blood sample, blood products, such as plasma, serum, and the like. Preferably the sample is from a subject and encompass fluids, solids, tissues, and gases.

As used herein, “blood sample” or “whole blood sample” encompasses a biological sample which is derived from blood obtained from an individual and can be used in a diagnostic or monitoring assay. The definition encompasses blood, plasma, and serum.

As used herein, a “reference value” can be an absolute value; a relative value; an average value; a median value, a mean value, or a value as compared to a particular control or baseline value. A reference value can be based on an individual sample or a large number of samples, such as from patients or normal individuals.

A “normalized measured” value refers to a measurement taken and adjusted to take background into consideration. Background subtraction to obtain total fluorescence is considered a normalized measurement. The background subtraction allows for the correction of background fluorescence that is inherent in the optical system and assay buffers.

### Diagnosing Blood Clotting Risks and Method of Treatment

In certain embodiment, this disclosure relates to methods of treating, preventing, or aiding in the diagnosis of patient having a risk of excessive bleeding. In certain embodiment, the patient is at risk of excessive bleeding due to a platelet disorder or autoimmune disorder. In certain embodiments, this disclosure relates to treatment methods comprising administering an effective amount of an agent that binds the Caf 1 domain or Caf 2 domain of the alpha subunit or the membrane-proximal-tail domain or any of the four epidermal growth factor (EGF)-like domains domain of the beta subunit of an αIIbβ3 integrin complex to a subject in need thereof.

In certain embodiments, this disclosure relates to methods of identifying, diagnosing, and treating a subject that is at a high risk of serious bleeding episodes, risk for severe bleeding, risk for major bleeding episodes, or at risk for life threatening hemorrhage using methods disclosed herein and treating a patient that is at risk of excessive bleeding with an agent that improves the ability of platelets to coagulate or form blood clots. In certain embodiments, an agent that improves the ability of platelets to coagulate or form blood clots is an agent that binds the Caf 1 domain or Caf 2 domain of the alpha subunit or the membrane-proximal-tail domain or any of the four epidermal growth factor (EGF)-like domains domain of the beta subunit of an αIIbβ3 integrin complex to a subject in need thereof.

In certain embodiments, this disclosure relates to methods increasing blood clotting comprising administering an effective amount of an agent that binds a Caf 1 domain or Caf 2 domain of the alpha subunit of an αIIbβ3 integrin complex to a subject in need thereof.

In certain embodiments, the subject is diagnosed with a low platelet count and a low platelet contraction force. In certain embodiments, the subject is diagnosed with a low contraction force using a contraction cytometer.

In certain embodiments, the subject is diagnosed with an autoimmune disease or inflammatory disorder. In certain embodiments, the subject is diagnosed with immune thrombocytopenia (ITP).

In certain embodiments, the agent is an antibody. In certain embodiments, the agent is an MBC.314.5 antibody or fragment thereof. In certain embodiments, the agent is administered in combination with a blood clotting agent. In certain embodiments, the agent is administered in combination with thrombin, fibrinogen, aprotinin, tranexamic acid (TXA), epsilon-aminocaproic acid, 4-(aminomethyl)benzoic acid (para-aminomethylbenzoic acid, PAMBA), or combinations thereof. In certain embodiments, the agent is administered topically in a composition further comprising a clotting agent, gelatin, cellulose, collagen, chitosan, aluminosilicate, albumin, thrombin, fibrinogen, or combinations thereof.

In certain embodiments, this disclosure relates to methods of increasing blood clotting comprising administering an effective amount of an agent that binds a membrane-proximal-tail domain or any of the four epidermal growth factor (EGF)-like domains of the beta subunit of an αIIbβ3 integrin complex to a subject in need thereof.

In certain embodiments, the subject is diagnosed with a low platelet count and a low platelet contraction force. In certain embodiments, the subject is diagnosed with a low contraction force using a contraction cytometer.

In certain embodiments, the subject is diagnosed with an autoimmune disease or inflammatory disorder. In certain embodiments, the subject is diagnosed with immune thrombocytopenia (ITP).

In certain embodiments, the agent is an antibody. In certain embodiments, the agent is an MBC 322.8, anti-LIBS-1, anti-LIBS-2 anti-LIBS3, anti-LIBS6 antibody, or fragment thereof. In certain embodiments, the agent is administered in combination with a blood clotting agent. In certain embodiments, the agent is administered in combination with thrombin, fibrinogen, aprotinin, tranexamic acid (TXA), epsilon-aminocaproic acid, 4-(aminomethyl)benzoic acid, or combinations thereof. In certain embodiments, the agent is administered topically in a composition further comprising a clotting agent, gelatin, cellulose, collagen, chitosan, aluminosilicate, albumin, thrombin, fibrinogen, or combinations thereof.

In certain embodiment, this disclosure relates to methods of treating, preventing, or aiding in the diagnosis of patient having a risk of excessive bleeding. In certain embodiment, the patient is at risk of excessive bleeding due to a platelet disorder or autoimmune disorder. In certain embodiments, this disclosure relates to treatment methods comprising administering an effective amount of an agent that binds the Caf 1 domain or Caf 2 domain of the alpha subunit or the membrane-proximal-tail domain or any of the four epidermal growth factor (EGF)-like domains of the beta subunit of an αIIbβ3 integrin complex to a subject in need thereof.

In certain embodiments, this disclosure relates to methods of identifying, diagnosing, and treating a subject that is at a high risk of serious bleeding episodes, risk for severe bleeding, risk for major bleeding episodes, or at risk for life threatening hemorrhage using methods disclosed herein and treating a patient that is at risk of excessive bleeding with an agent that improves the ability of platelets to coagulate or form blood clots. In certain embodiments, an agent that improves the ability of platelets to coagulate or form blood clots is an agent that binds the Caf 1 domain or Caf 2 domain of the alpha subunit or the membrane-proximal-tail domain or any of the four epidermal growth factor (EGF)-like domains of the beta subunit of an αIIbβ3 integrin complex to a subject in need thereof.

In certain embodiment, this disclosure relates to methods of treating or preventing excessive bleeding in a patient that is at risk of excessive bleeding comprising administering an effective amount of an agent that binds the Caf 1 domain or Caf 2 domain of the alpha subunit of an αIIbβ3 integrin complex to a subject in need thereof. In certain embodiment, agent is an antibody such as MBC.314.5.

In certain embodiment, this disclosure relates to methods of treating or preventing excessive bleeding in a patient that is at risk of excessive bleeding comprising administering agent that binds the membrane-proximal-tail domain or any of the four epidermal growth factor (EGF)-like domains of the beta subunit of an αIIbβ3 integrin complex. In certain embodiment, the agent is an antibody such as MBC 322.8, anti-LIBS-1, anti-LIBS-2 anti-LIBS3 and anti-LIBS6.

In certain embodiments, this disclosure relates to methods of diagnosing or identifying a human patient with a bleeding risk comprising: providing contraction cytometer as disclosed herein, e.g., comprising a solid substrate comprising, a deformable polyacrylamide hydrogel, a plurality of fibrinogen pairs with a first fibrinogen zone and a second fibrinogen zone separated by distance on the deformable polyacrylamide hydrogel, and a fluorescent dye, label, or other signal generator conjugated to the first and second fibrinogen zones; contacting a sample from a patient with the contraction cytometer, wherein the sample comprises a platelet that adheres to one of the of first and/or second fibrinogen zones on the solid substrate; measuring the distance of between the first and second zones; correlating the distance to a quantitative contraction force, e.g., low contraction force associated with no or small average change in position and a higher contraction force associated with larger changes, or exceeding a threshold contraction force, or a normal contraction force; and correlating a low contraction force as indicative of a high risk of bleeding for the patient or correlating a quantitative value that exceed a threshold contraction force as indicative that the patient is at a lower risk of excessive bleeding. In certain embodiments, correlating the distance or change of distance (indicated by measuring the location of the label) to a quantitative contraction force can be done by comparing a measured value to a normal or reference value.

In certain embodiments, this disclosure relates to a platelet contraction cytometer comprising a micropatterned surface having pairs of first and second zones that exceed 1,000, 10,000 pairs, or 100,000 per cm², wherein the first and second zones comprise fibrinogen for use in measuring platelet contraction forces of less than 25 nN or 40 nN (nanoNewtons).

In certain embodiments, a micropatterned surface comprises pairs of first and second zones that exceed 100,000 pairs per cm², wherein the first and second zones comprise fibrinogen for use in measuring platelet contraction forces of less than 25 nN.

In certain embodiment, this disclosure relates to methods using a contraction cytometer to determine that a patient does not have a high risk of excessive bleeding and proceed to perform a medical procedure that poses a risk of bleeding such as including cardiovascular surgeries, hepatic surgeries, orthopedic surgeries, and spinal surgeries.

In certain embodiment, this disclosure relates to methods of using the contraction cytometer to determine that a patient does have a high risk of excessive bleeding and proceed to perform a medical procedure that poses a risk of bleeding such as including cardiovascular surgeries, hepatic surgeries, orthopedic surgeries, and spinal surgeries, and thereafter administering a higher dose than average dose of a clotting agent, or administering a dose at an earlier time than normal, e.g., considering the weight of the patient, that promotes blood clotting earlier.

In certain embodiments, measuring contraction between the two zoned is accomplished by detecting the signal generator distance or changed of distance (indicated by measuring the location of the label), wherein the platelet adheres and pulls the first and second fibrinogen zones together closer in distance wherein the contractile force is proportional to first and second zone displacement.

In certain embodiment, the low contraction force is less than 40 nN, 30 nN, 25 nN, or 20 nN and wherein the patient has a platelet count of less than 40 k/microliter or 25 k/microliter of blood.

In certain embodiment, the high risk of bleeding is risk of major bleeding or life-threatening hemorrhage.

In certain embodiment, the signal generator is a fluorescence microscope that detects a fluorescent dye conjugated to the first and second fibrinogen zones.

In certain embodiments, the patient is diagnosed with an autoantibody mediated platelet disorder.

The method of claim the autoantibody mediated platelet disorder is immune thrombocytopenia (ITP).

In certain embodiment, the human patient is diagnosed with a platelet count of less than 100×10⁹/L.

In certain embodiments, this disclosure relates to methods of identifying a patient with a high bleeding risk comprising detecting auto antibodies in the patient, detecting that the auto antibodies bind αIIbβ3 integrin complex that results in the autoantibody αIIbβ3 integrin complex bound having a compacted structure. In certain embodiment, the compacted structure is a bent or extended closed confirmation in the αIIbβ3 integrin complex.

### Autoantibodies Immuno-Mechanically Modulate Platelet Contractile Force and Bleeding Risk

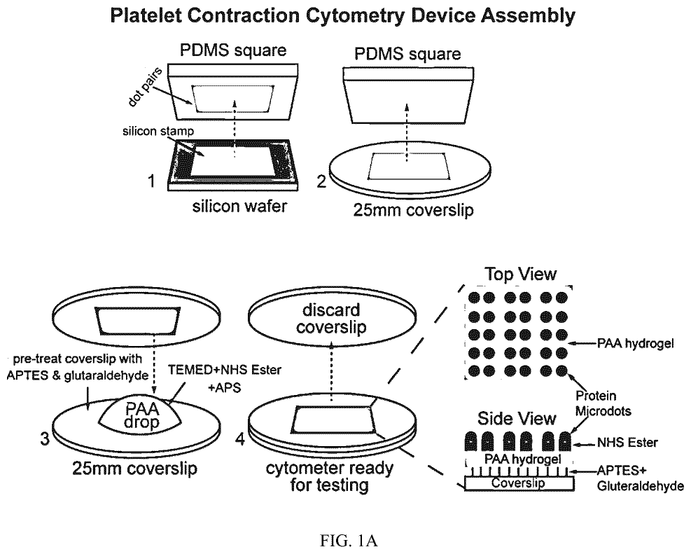

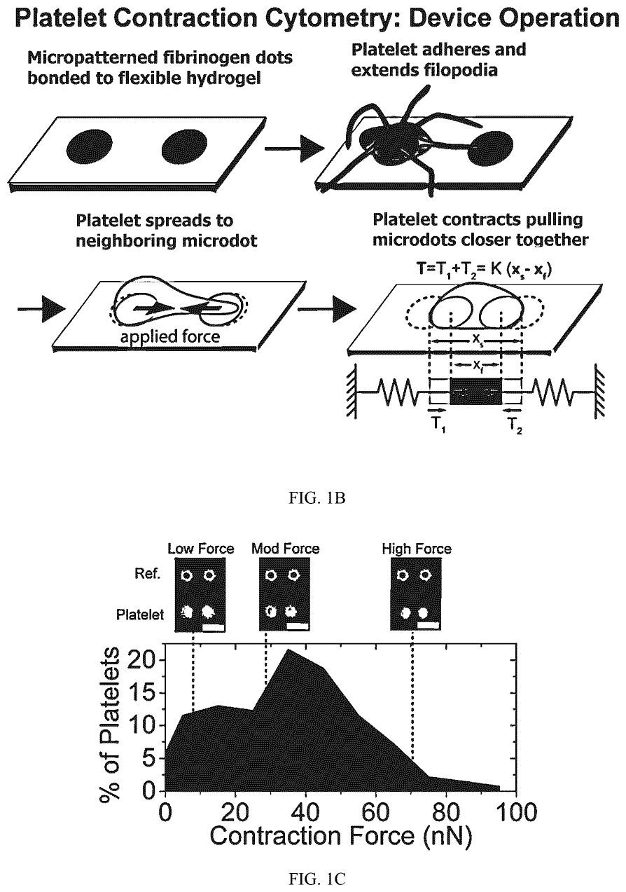

The cellular contraction cytometer contains a hydrogel-based micropatterned coverslip useful for translational research (FIG. 1A) allowing one to generate force cytometry data. The surface of the hydrogel-coverslip is micropatterned with approximately 1 million pairs of fluorescently labeled “microdots” bio-conjugated with extracellular matrix. Cells in suspension incubated with the system attach to the microdot pairs and contract the dots together. The applied cellular contractile force is directly proportional to the displacement of the dots (FIG. 1B). To use the system, a cell suspension is simply placed atop and incubated onto the hydrogel-laden coverslip and imaged, which can be measured for each instance with fluorescence microscopy. The hydrogel laden coverslip provides streamlined assembly improving the production speed.

Using a micropatterned hydrogel-laden coverslip compatible with standard fluorescence microscopy, a clinical mechanobiology study was conducted, specifically focusing on immune thrombocytopenia (ITP), an autoantibody-mediated platelet disorder that currently lacks a reliable biomarker for bleeding risk. In ITP patients (n=49), low single platelet contraction force alone is a “physics-based” biomarker of bleeding (100% sensitivity, 89.5% specificity). Mechanistically, autoantibodies and monoclonal antibodies drive increases and decreases of cell force by stabilizing integrins in different conformations depending on the targeted epitope. Hence, immuno-mechanical modulation demonstrates how antibodies can pathologically alter mechanotransduction to cause clinical symptoms. This phenomenon can be leveraged to control cellular mechanics for research, diagnostic, and therapeutic purposes.

As platelets adhere, spread, and contract in this system, whether these observed decreases in force correlate with a concomitant decrease in platelet adhesion and spreading was investigated. Standard platelet adhesion and spreading assays were performed on fibrinogen coated glass surfaces and healthy donor platelets were treated with the epitope specific monoclonal antibodies. There was little correlation between platelet force, platelet adhesion, and platelet spreading. Some antibodies increased platelet force yet had a negligible effect on platelet adhesion. These biophysical behaviors can be independently or semi-independently modulated and there exists a more nuanced effect of antibodies on platelet biophysical behavior.

Antibodies that competitively bind, and therefore have closely adjacent or overlapping epitopes, can induce different changes to downstream biophysical behaviors. For example, abciximab and PAC-1 both target the binding pocket of αIIbβ3, yet PAC-1 lowers contractile forces whereas abciximab blocks adhesion. Similarly, 10E5 and MBC 290.5 bind to similar locations, yet MBC 290.5 leads to lower forces whereas 10E5 completely blocked the ability of platelets to spread to the neighboring microdot. As such, slight differences in the epitopes that the antibodies bind lead to stark differences in their biophysical and modulatory effects.

By leveraging a high-throughput contraction cytometer one is able to use a coverslip-based technology that can be adapted to standard microscopy for measuring numerous single cell platelet force (greater than 10,000 measurements). The high-throughput contraction cytometer provides a direct link between cellular mechanical measurements at the single cell level and patient phenotype in ITP.

Here, a clinical biomarker is contemplated, one that is biophysical nature. This disclosure provides for methods to identify decreased platelet contractile forces that are associated with bleeding in the setting of an autoimmune disease. Single cell biophysics is contemplated to predict clinical outcomes. Systems disclosed herein allow for single cell measurements of force at high throughput. Mechanistic underpinnings of decreased platelet force in ITP are describe. The cause and effects of how decreased platelet contraction may lead to bleeding in ITP is provided.

It is believed that this is the first study linking integrin conformation to a specific clinical pathology, where the immune system of a patient by way of antibodies can modulate the biophysical behavior of their cells causing a clinical phenotype depending on the integrin confirmation and epitopes the antibodies bind. These discoveries points to a possible protective effect from the autoantibodies, where increased platelet strength counters the low platelet count. Such an approach is contemplated as a therapeutic strategy. It is contemplated that varying antibody binding locations one can modulate integrin conformational flexibility.

### Cell Contraction Force is a Clinical Biophysical Biomarker

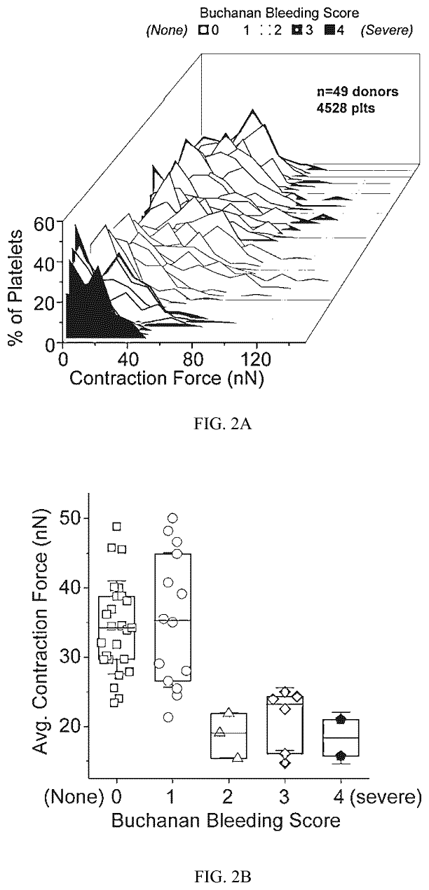

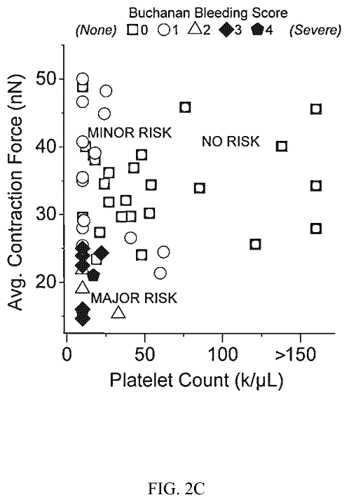

Data indicates that ITP patient samples have low platelet contractile forces strongly correlate with bleeding severity and is therefore a potential biophysical biomarker of bleeding. Patient bleeding was quantified with the Buchanan bleeding score, which relies on a variety of visual markers to grade patient bleeding from 0 (none) or 1 (minor) to 4 (severe) or 5 (life-threatening). Platelets from ITP patients with higher bleeding scores consistently lack highly contractile subpopulations of platelets (FIG. 2A). When the platelet contractile force of platelets from sample of a patient is averaged, significant differences between the average platelet contractile force were observed in patients with bleeding scores less than 2 compared to patients with bleeding scores about or greater than 2. Furthermore, platelets from patients with bleeding scores of 1 or 0 exerted average contractile forces of approximately 35 nN. In contrast, platelets from patients with higher bleeding scores of 2, 3, 4 exhibited average contractile forces of 18.8 nN, 21 nN, and 18.4 nN, respectively (FIG. 2B). Both platelet count and mean platelet volume were evaluated as possible biomarkers of bleeding. However, neither platelet count nor platelet volume provide substantial stratification of patients with different Buchanan bleeding scores. Platelet counts greater than 40 k/μL seem to greatly decrease bleeding risk. Additionally, with a regression tree and only leveraging platelet contraction cytometry, average platelet contractile forces of less than about 25 nN optimally predict bleeding scores of less than or equal to 2 with a clinical sensitivity of 100% and a specificity of 89.5%. Upon further analysis, it was found that using both platelet force and platelet count as biomarkers further improves prediction of moderate to severe bleeding scores. While four ITP patients with average platelet contraction forces less than 25 nN exhibited little to no bleeding, three of these patients had platelet counts greater than 40 k/μL. To that end, when platelet count and platelet contraction force are plotted together, patients can be stratified into having different bleeding risks. Patients with a bleeding score of about or greater than 2 often had platelet contractile forces less than 25 nN and platelet counts of less than 40 k/μL, suggesting that low numbers in both parameters is indicative of a “major risk” of bleeding. Patients with either low platelet contractile force or low platelet count had a “minor risk” of bleeding, and consistently exhibited bleeding scores of 0 to 1. The diagnostic value of platelet contraction force, platelet volume and platelet count alone were examined in addition to both platelet volume and count in conjunction with platelet contractile force via a receiver operator characteristic (ROC) analysis, which enables visualization of combined sensitivity/specificity as the area under the curve (AUC). Between platelet contractile force, platelet count, and mean platelet volume (MPV), platelet contractile force was the strongest predictor of bleeding, with an AUC of 0.97 (with AUC=1 being a “ideal” test). Platelet count and MPV were associated with lower AUCs of 0.8 and 0.64, respectively, whereas an AUC of 0.99 was achieved by combining all three biomarkers together.

Longitudinal studies were performed in a subset of the ITP patient cohort. The results indicated that changes in platelet contractile force correlated with the appearance or resolution of bleeding symptoms and that the platelet contractile force of an individual can change over time. Specifically, serial measurements from seven ITP patients indicated increases in platelet contractile force and platelet count were associated with reduced bleeding, while decreases in platelet contractile force and platelet count were associated with increased bleeding. Collectively, these data raised the possibility that an extrinsic factor which changes over time was modulating platelet contractile force in ITP.

### Antibodies Modulate Single Cell Contraction Force

Upon identifying that low platelet contractile force is the only major difference between ITP patients with bleeding compared to those without, studies were conducted to investigate the underlying mechanisms. Autoantibodies are known to target platelet antigens, predominantly targeting αIIbβ3 (70-80%), GP1b complex (20-40%) or both. Platelet destruction then occurs via antibody-mediated destruction in the spleen via macrophages in Fc-dependent manner or Fc-independent desialyation and platelet clearance in the liver via hepatocyte Ashwell-Morell receptors. However, using autoantibodies for diagnostic purposes has been historically problematic. Autoantibodies are often undetectable in ITP patient plasma and even autoantibodies that are detectable exhibit little correlation to bleeding, which was confirmed by sending patient platelet poor plasma (PPP) samples with varying bleeding scores to a diagnostic laboratory to undergo a platelet antibody screen.

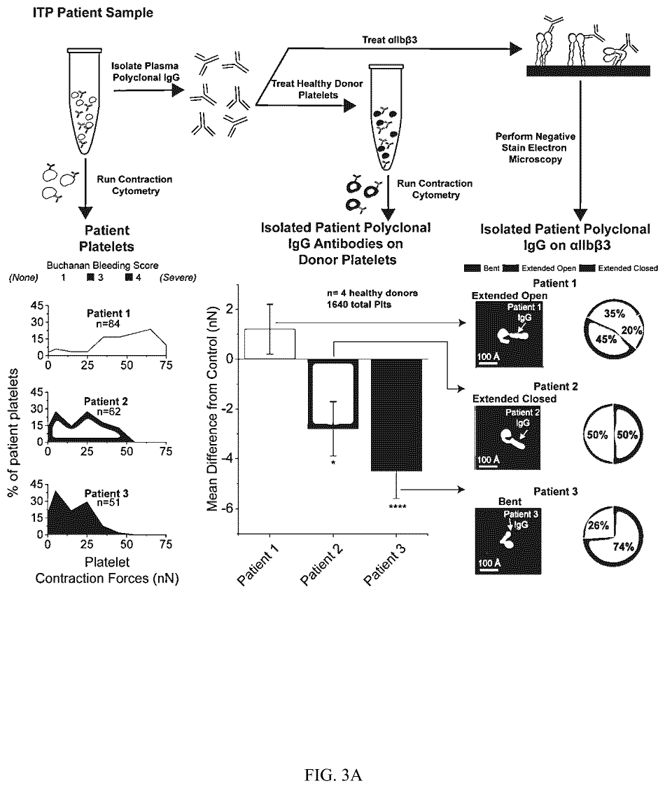

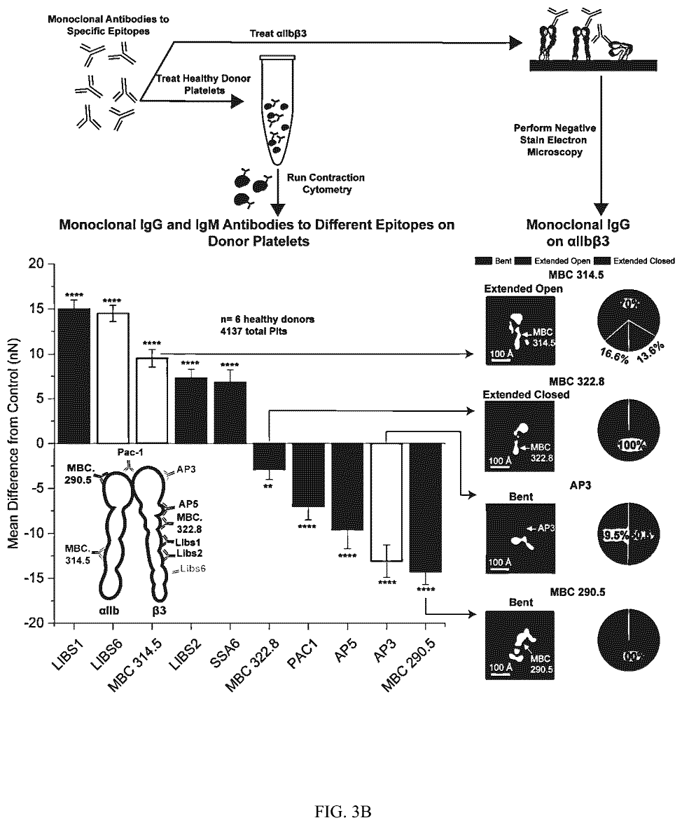

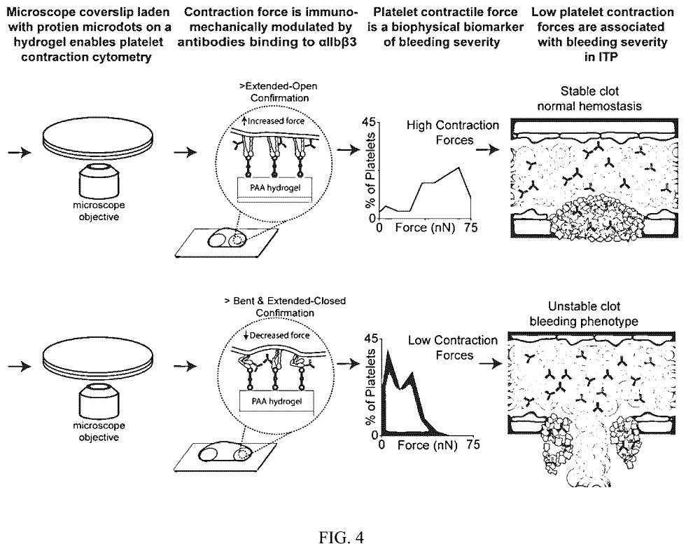

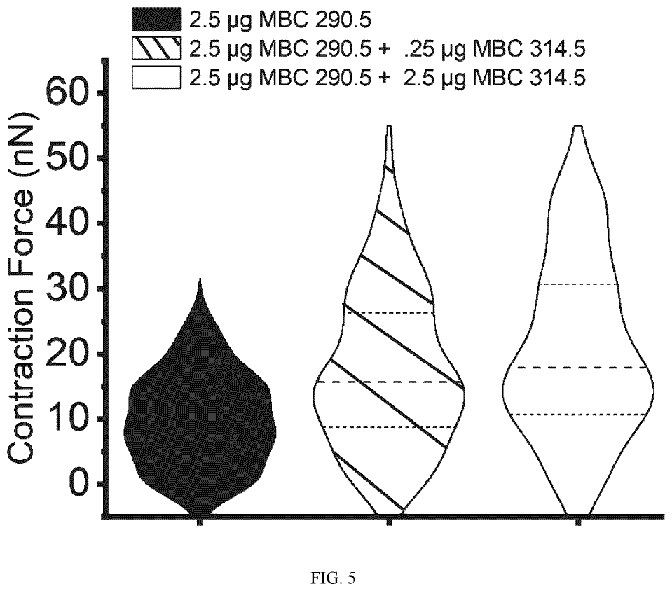

Experiments indicated that polyclonal IgG platelet-associated (PA) antibodies isolated from ITP patients (FIG. 3A) directly modulate the contractile forces of healthy donor platelets in an integrin conformation dependent manner. Using platelet poor plasma (PPP) from 3 patients confirmed to possess IgG platelet reactive antibodies, IgG antibodies were isolated and incubated on to healthy donor platelets. Antibodies derived from the PPP of symptomatic ITP patients with higher bleeding scores of 3 and 4 decreased contractile forces of platelets from healthy donors by 17% and 10%, respectively. Antibodies isolated from an ITP patient with high platelet contractile forces and a low bleeding score of 1 directly increased platelet contractile force of healthy donor platelets by 4%, although this was not statistically significant.

As the majority of PA antibodies in ITP are directed against the integrin αIIbβ3. Because the system uses fibrinogen, the ligand for αIIbβ3, negative stain electron microscopy (EM) was performed to determine if the PA antibodies from ITP patients bind to this integrin. Anti-αIIbβ3 antibodies were detectable in ITP patient samples used for these experiments. The antibodies bound to and stabilized integrins into different conformations. Patients with bleeding symptoms and lower platelet contractile forces had antibody-integrin complexes in only the bent and extended closed conformations. In contrast, patients with high forces and a low bleeding score had a substantial population of antibody-integrin complexes in the extended-open conformation. The clinical ramifications of knowing that antibodies bind and stabilize integrin conformations suggest the usefulness of therapeutic antibodies for managing pathophysiology of a disease process. PA antibodies can immuno-mechanically modulate platelet contractile force, both up and down, in an integrin conformation and epitope-dependent manner, which ultimately correlates to a bleeding severity in ITP patients.

Experiments were performed to better understand the relationship between conformation and epitope-dependent platelet contractile force modulation with well-characterized monoclonal antibodies targeted to different epitopes of αIIbβ3 (FIG. 3B). Experiments indicate that it was possible to modulate platelet contractile forces both up and down, from a maximum increase of 57% (+15 nN) to maximum decrease of 55% (−15 nN). Monoclonal antibodies that bind toward the head region decrease platelet contractile force, while antibodies that bind to the tail region increase platelet contractile force. With regard to antibody-integrin conformation and total contractile force, negative-stain EM studies with monoclonal antibodies demonstrated identical trends as the polyclonal ITP patient antibodies (FIG. 3B). Antibodies binding and stabilizing the bent conformation dramatically decrease platelet contractile force, whereas antibodies stabilizing the extended-closed slightly decrease force, and antibodies stabilizing the extended-open conformation dramatically increase force. Taken together, the monoclonal and polyclonal data suggest that integrin conformation is the driver of single platelet force, further confirming that antibodies can bind to integrins on cells and immuno-mechanically modulate cellular biophysical behavior.

### Platelet Preparation

Blood was drawn into acid-citrate-dextrose (ACD) solution 2 (VWR) and was subsequently centrifuged at 150 G for 15 min without brake and the resulting platelet rich plasma (PRP) was centrifuged again with an additional 10% ACD by volume at 900 G for 5 min without brake. The supernatant, platelet poor plasma, was discarded or utilized to isolate patient polyclonal IgG antibodies. The platelet pellet was resuspended into HEPES modified buffer and was gel filtered into this same buffer using a gel filtration column. Platelets were then diluted to 2 million/mL prior to contraction cytometry to reduce paracrine signaling and minimize platelet aggregate formation.

### Platelet Contraction Cytometer Device Fabrication

Plasma treated circular coverslips (25 mm Circle No. 1) were incubated in a 10% (3-Aminopropyl) trimethoxysilane/90% Ethanol/0.01% Glacial acetic acid solution for 90 min at 60 degrees Celsius. The coverslip was then rinsed 3 times with 70% ethanol and then rinsed 3 times with DI water. The coverslips were then treated with a 2% glutaraldehyde solution for 30 minutes at room temperature. After treatment, coverslips were rinsed with DI water and dried with nitrogen gas.

Alexa Fluor 488 tagged fibrinogen was incubated on square (10 mm×10 mm×3 mm) polydimethylsiloxane (PDMS) at 30 μg/mL for 30 minutes at 37 degrees Celsius before being rinsed with water and dried with nitrogen gas. The fibrinogen coated PDMS squares were then placed on to a silicon mold and lifted off to create a micropatterned PDMS “stamp. “The silicon mold was created using etching and lithography techniques. Then the newly made PDMS stamp was then placed on to a plasma treated circular coverslip (25 mm Circle No. 1) and then lifted off to transfer the microdot pattern to the coverslip.

The assembly of the platelet contraction cytometer was performed in a nitrogen filled glovebox. The glutaraldehyde treated coverslip and micropatterned coverslip were placed in the glove box antechamber and vacuumed prior to assembly in the glove box. Pre-mixed polyacrylamide hydrogels were mixed with N,N,N′,N′-Tetramethylethylenediamine (TEMED), ammonium persulfate, and acrylic acid N-hydroxysuccinimide ester (NHS28 ester) and 40 μL of this hydrogel solution was pipetted onto each glutaraldehyde treated coverslip. A glove helps speed up the polymerization and allows for lower concentrations of APS, TEMED and NHS-Ester to be used. The micropatterned coverslip was then inverted on to the hydrogel solution immediately after, creating a “sandwich” and the hydrogel was allowed to polymerize in the glovebox for 90 minutes to ensure complete polymerization. After polymerization, the hydrogel “sandwich” was removed from the glove box and the top micropatterned coverslip was removed and discarded as the micropattern is now transferred to the top of the formed hydrogel, this creating the contraction cytometer. The newly formed contraction cytometers were stored for up to 7 days in PBS at 4 degrees Celsius in petri dishes.

### Platelet Contraction Cytometer Device Operation

Each contraction cytometer contains large arrays of fibrinogen microdots pairs that are patterned onto a polyacrylamide hydrogel with a stiffness of about 75 k Pa. Each microdot pair possesses 2 microdots with a radius of about 0.8 μm and a separation of about 4 μm. These values for microdot size and spacing ensured that platelets preferentially attached to the microdots and were able to spread to neighboring microdot in the microdot pair and contract. Once fabricated, 2 million/mL platelets, 3 mM CaCl₂), 3 mM MgCl₂ and 1 U/mL thrombin were then plated on to the contraction cytometer for 90 minutes and allowed to displace the microdot pairs. Similar to a spring, the fibrinogen microdots displacement is proportional to the contraction force.

Platelet contraction was imaged on a confocal microscope using a 20×/0.8 NA lens. Platelets were tagged with cell mask orange 554/567 nm. Images were analyzed using a script which calculated the center-to-center distance between fibrinogen microdots. The microdot displacement produced by each contracting platelet was compared to an uncontracted reference dot pair. Contracted platelets are identified and selected for analysis. Traction forces (T) of individual platelets were calculated as: T=2πGa (xₛ−x_{f})/2−v. G is the shear modulus, a is the microdot radius, v is Poisson's ratio and xₛ is the starting distance, and x_{f} is the final distance post contraction.

### Patient Polyclonal IgG Isolation and Contraction Experiments

ITP patient platelet poor plasma samples with varying bleeding scores were sent to a vendor to undergo platelet antibody screen. All positive samples had platelet reactive IgG antibodies and not IgM. As such, IgG antibodies from 3 patient samples with varying bleeding scores were isolated. Healthy donor platelets were treated with 10 μg/mL of patient polyclonal IgG antibodies for 15 minutes prior to platelet activation with thrombin and incubation on the contraction cytometer.

### Monoclonal Antibody Contraction Experiments

Healthy donor platelets were treated with 2.5 μg/mL of well characterized monoclonal antibodies 15 minutes prior to platelet activation with thrombin and incubation on contraction cytometer. Antibodies MBC 290.5, AP3, AP5, MBC 322.8, MBC 314.5, PAC-1, HIP8, LIBS2 Abciximab, and 10E5 were purchased from commercial vendors. LIBS1 and LIBS6 were gift from Mark Ginsberg. SSA6 was a gifted from Joel Bennett's lab.

### Monoclonal Antibody Negative Stain Electron Microscopy

Recombinant human Integrin αIIbβ3 complexed with monoclonal antibody was diluted to 0.04 mg/ml in PBS before grid preparation. A 3 μL drop of diluted protein was applied to previously glow-discharged, carbon-coated grids for about 60 sec, blotted and washed twice with water, stained with 0.75% uranyl formate, blotted and air-dried. Between 50- and 70 images were collected on a microscope at 73,000 magnification and 1.97 Å pixel size.

### Patient Polyclonal Antibody and Integrin αIIbβ3 Complex Preparation and Purification

To prepare the Integrin αIIbβ3 protein polyclonal antibody complexes, patient polyclonal IgG was purified using a protein-A capture resin. Purified IgG was subjected to papain mediated proteolytic digestion for Fab fragmentation. Fab was further purified away from undigested IgG and Fc by incubation with protein-A agarose beads. Purified Fab was incubated with Integrin αIIbβ3, at molar ratio of 20:1, followed by purification of Fab bound antigen complexes using size exclusion chromatography. Fractions corresponding to Integrin αIIbβ3 protein polyclonal antibody complex was used for subsequent sample preparation. Imaging and data analysis was done as above by negative stain-EM.

Platelet Adhesion Assays with Monoclonal Antibodies

Platelets were diluted to 10×10⁹/L to ensure the measurement of single platelets and then incubated with selected monoclonal antibody for 15 minutes prior to experimentation. Antibody treated platelets were then incubated on coverslips on 100 μg/mL human fibrinogen and allowed to adhere for 2 hours. Platelets were imaged with an inverted microscope using a 40×/1.30 NA lens and then analyzed for adhesion density and spreading area using developed software.

1. A method of increasing blood clotting comprising administering an effective amount of an agent that binds a Caf 1 domain or Caf 2 domain of the alpha subunit of an αIIbβ3 integrin complex to a subject in need thereof.

2. The method of claim 1, wherein the subject is diagnosed with a low platelet count and a low platelet contraction force.

3. The method of claim 1, wherein the subject is diagnosed with a low contraction force using a contraction cytometer.

4. The method of claim 1, wherein the subject is diagnosed with an autoimmune disease or inflammatory disorder.

5. The method of claim 1, wherein the subject is diagnosed with immune thrombocytopenia (ITP).

6. The method of claim 1, wherein the agent is an antibody.

7. The method of claim 1, wherein the agent is an MBC.314.5 antibody or fragment thereof.

8. The method of claim 1, wherein the agent is administered in combination with a blood clotting agent.

9. The method of claim 1, wherein the agent is administered in combination with thrombin, fibrinogen, aprotinin, tranexamic acid (TXA), epsilon-aminocaproic acid, 4-(aminomethyl)benzoic acid, or combinations thereof.

10. The method of claim 1, wherein the agent is administered topically in a composition further comprising a clotting agent, gelatin, cellulose, collagen, chitosan, aluminosilicate, albumin, thrombin, fibrinogen, or combinations thereof.

11. A method of increasing blood clotting comprising administering an effective amount of an agent that binds a membrane-proximal-tail domain or any of the four epidermal growth factor (EGF)-like domains of the beta subunit of an αIIbβ3 integrin complex to a subject in need thereof.

12. The method of claim 11, wherein the subject is diagnosed with a low platelet count and a low platelet contraction force.

13. The method of claim 11, wherein the subject is diagnosed with a low contraction force using a contraction cytometer.

14. The method of claim 11, wherein the subject is diagnosed with an autoimmune disease or inflammatory disorder.

15. The method of claim 11, wherein the subject is diagnosed with immune thrombocytopenia (ITP).

16. The method of claim 11, wherein the agent is an antibody.

17. The method of claim 11, wherein the agent is an MBC 322.8, anti-LIBS-1, anti-LIBS-2 anti-LIBS3, anti-LIBS6 antibody, or fragments thereof.

18. The method of claim 11, wherein the agent is administered in combination with a blood clotting agent.

19. The method of claim 11, wherein the agent is administered in combination with thrombin, fibrinogen, aprotinin, tranexamic acid (TXA), epsilon-aminocaproic acid, 4-(aminomethyl)benzoic acid, or combinations thereof.

20. The method of claim 11, wherein the agent is administered topically in a composition further comprising a clotting agent, gelatin, cellulose, collagen, chitosan, aluminosilicate, albumin, thrombin, fibrinogen, or combinations thereof.

21. A micropatterned surface comprising pairs of first and second zones that exceed 100,000 pairs per cm², wherein the first and second zones comprise fibrinogen for use in measuring platelet contraction forces.
